(19) United States
(12) Patent Application Publication
(10) Pub. No.: US 2006/0019405 A1
(43) Pub. Date: Jan. 26, 2006

(54) Cat lavator comprising an urine test

(75) Inventor: Gunnar Wenzel, Lohmar (DE)

Correspondence Address: FULBRIGHT & JAWORSKI, LLP, 1301 MCKINNEY, SUITE 5100, HOUSTON, TX, 77010-3095, US

(73) Assignee: MARS INCORPORATED, MCLEAN, VA (US)

(21) Appl. No.: 10/520,440

(22) PCT Filed: Jul. 8, 2003
(86) PCT No.: PCT/EP03/07296

§ 371 Date: Jun. 24, 2005

(30) Foreign Application Priority Data
Jul. 19, 2002 (DE) 102 32 905.2

(51) Int. Cl. G01N 33/558; C12Q 1/54

(52) U.S. Cl. 436/514; 435/14

(57) ABSTRACT

The present invention relates to a cat lavatory including at least one absorbent material comprising an insert of non-woven material, at least one indicator strip for determining a cat's urine levels, which is arranged on and/or in the absorbent material; and a method of determining a cat's urine levels using the cat lavatory of the invention.

### CROSS-REFERENCE TO RELATED APPLICATIONS

This Application is a National Stage of application PCT/EP03/07296 filed on Jul. 8, 2003 claiming priority to DE 10232905.2 filed Jul, 19, 2002.

### TECHNICAL FIELD

The present invention relates to a cat lavatory comprising at least one absorbent material comprising an insert of non-woven material, and a method of determining a cat's urine levels using the same.

### BACKGROUND OF THE INVENTION

Cat lavatories are sufficiently well-known in the art. Conventional cat lavatories usually consist of a tray-like lower part, which is typically filled with absorbent material, especially cat litter. Some or all of the absorbent material must be removed from the lower part at regular intervals and replaced with new material. In addition, it is necessary, for reasons of hygiene, also to clean the lower part at regular intervals.

Disposable cat lavatories are also known from the prior art, in which, for example, a bag filled with cat litter is placed in the lower part, the bag being sealed after use and disposed of in its entirety.

DE 36 25 528 C2, for example, teaches a cat lavatory consisting of a soft disposable pack made of a flexible material with a receptacle space closed on all sides and containing a filling of hygienic litter, wherein the space can be torn open at the top in order for the pack to be used after use, the pack containing the litter material can be disposed of in a sealed condition.

The sole function of all the cat lavatories known from the prior art, however, is to take up the cat's faeces and urine in a highly absorbent manner and to bind the odours.

In many cases, however, there is an interest, especially if the cat is ill, in checking a cat's true state of health. Apart from examining its blood count, this can be done above all by determining urine levels, such as the sugar content or any possible blood content in the urine. Appropriate test strips for examining selected urine levels are known for human use. It would, however, appear that a corresponding use in a cat is difficult and, in particular, it would inevitably involve the need for the cat owner using it to come into contact with the soiled lavatory, which is unhygienic and undesirable.

The object of the present invention is therefore to achieve a further development of the generic cat lavatory such that the disadvantages of the prior art are overcome, and in particular that it becomes possible to determine the cat's urine levels in an hygienic and simple manner.

In addition, a further object of the invention consists in providing a method of determining a cat's urine levels using the cat lavatory of the invention.

### BRIEF SUMMARY OF THE INVENTION

The first object is achieved by at least one indicator strip for determining a cat's urine levels, which is arranged on and/or in the absorbent material.

In a preferred embodiment of the invention, the cat lavatory consists of a container with a peripheral side wall, which is sealed on all sides in the condition in which it is delivered to the consumer, and which contains absorbent material, and which is designed in such a way that it is opened before use and is disposed of in its entirety after use.

In addition, by way of alternative, the cat lavatory can consist of a lower part and a replaceable pouch containing the absorbent material, which can be placed in the lower part.

It is further preferred for the insert of non-woven material to comprise a super-absorber and a scratch-proof layer of non-woven material disposed on top of the super-absorber.

Furthermore, a layer of litter can be disposed above the scratch-proof layer of non-woven material.

In a further preferred embodiment of the invention, the indicator strip is disposed between the super-absorber and the scratch-proof layer of non-woven material.

In a still more preferred embodiment of the invention, the indicator strip comprises at least one indicator field and a region for the user to handle the indicator strip.

In addition, it can be provided for the indicator field to comprise at least one indicator point.

It is especially preferred for the indicator field, in the condition in which it is delivered to the consumer, to be arranged within a protective cover.

It is especially preferred for the protective cover to be made substantially from water-proof plastic material and to be attached on and/or in the absorbent material.

The protective cover is preferably attached on and/or in the absorbent material with spots of adhesive.

Preferably, the indicator strip is substantially made from water-proof plastic material.

In addition, the indicator strip can be fixed to the absorbent material with at least one further attachment means.

In accordance with the invention, the indicator strip can be activated by pulling it out over the absorbent material.

In a preferred embodiment of the invention, the indicator field is disposed on the side of the indicator strip facing the super-absorber.

The method object of the invention is achieved by the fact that, in order to activate the indicator strip, it is drawn over the insert of non-woven material, especially the super-absorber.

In one embodiment, the indicator strip can be used to determine urine levels, such as sugar and blood contents and the like.

The present invention is thus based on the surprising finding that, by arranging an indicator strip on and/or in the absorbent material of the cat lavatory, a diagnostic function is provided which makes it possible to check certain urine levels in a cat in a simple and hygienic manner. A particular advantage of the cat lavatory of the invention is that it is not necessary for the user to come into contact with the soiled lavatory in order to perform measurements.

### BRIEF DESCRIPTION OF THE DRAWINGS

Further features and advantages of the present invention will become apparent from the following description, in which a preferred embodiment of the cat lavatory of the invention will be described with reference to schematic drawings, in which:

FIG. 1 shows a schematic view of a section of a cat lavatory in accordance with the invention; and

FIG. 2 shows a perspective view of a cat lavatory in accordance with the invention.

### DETAILED DESCRIPTION OF THE INVENTION

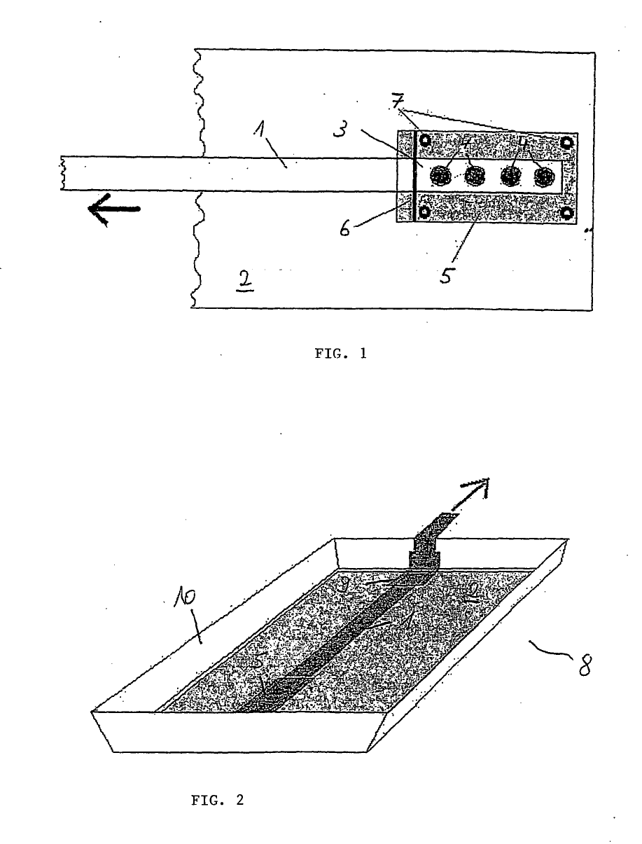

FIG. 1 shows an indicator strip 1 disposed on and/or in absorbent material 2, which consists in particular of a super-absorber and an insert of non-woven material. The indicator strip 1 comprises an indicator field 3, which consists of four indicator points 4. It goes without saying that the indicator region can also comprise one single large field or some other number of indicator points 4. In the condition in which it is delivered to the consumer, the indicator field 3 of the indicator strip 1 is disposed, as shown in FIG. 1, within a protective cover 5 which seals off the indicator field 3 from the absorbent material 2 with a seal 6. Like the indicator strip 1, the protective cover 5 is preferably made from a plastic material, and most preferably from a water-proof plastic material. The protective cover 5 is attached to the absorbent material 2, especially the layer of non-woven material, with adhesive points 7, for example.

FIG. 2 shows a preferred embodiment of the cat lavatory in accordance with the invention, in a perspective view. As can be seen, the indicator strip 1 is placed centrally in a cat lavatory 8. It goes without saying for a man skilled in this art that different arrangements of the indicator strip 1 and also that a plurality of indicator strips can be used. At a position spaced apart from the protective cover 5 of the indicator field 3, the indicator strip 1 shown in FIG. 2 is provided with an additional attachment 9, which ensures that the indicator strip 1 remains stably in position and which prevents the indicator strip 1 from slipping out of position and possible being rendered unusable when the cat lavatory 8 is used, such as if the cat scratches it with its paws. As can be seen from FIG. 2, the indicator strip 1 is slipped movably through the attachment 9, so that it can be drawn out in the direction of the arrow.

According to FIG. 2, the indicator strip 1 is disposed in a cat lavatory 8, which comprises a container with a peripheral side wall 10. This cat lavatory 8 can be a conventional cat lavatory, it then only being necessary to replace absorbent material 2 from time to time.

Similarly, the cat lavatory 8 shown in FIG. 2 can also be a disposable cat lavatory, which, in the condition in which it is delivered to the consumer, is sealed on all sides, is optionally folded, and which contains absorbent material 2, and is designed in such a way that the container is opened before use and is disposed of in its entirety after use.

It is also conceivable that only a replaceable pouch, which can be placed in the lower part, might be placed in the lower part, it being possible for a suitable indicator strip 1 to be located on said pouch.

It goes without saying that the cat lavatory 8 of the invention can also be provided with a corresponding top part (not shown), in order where appropriate to stabilise the cat lavatory 8 or corresponding inserts, such as disposable lavatories. In the case of disposable lavatories, it is also possible for sealing means to be provided for sealing the disposable lavatory after use.

The indicator strip 1 which is shown in FIGS. 1 and 2 is now used in such a way as to be able to read off the corresponding urine levels by pulling out the indicator strip 1 in the direction of the arrows shown in FIGS. 1 and 2 and/or drawing it over the absorbent material 2, especially the super-absorber, which in effect serves as a urine collector. In the process, first of all the seal 6 is broken open, and the indicator points 4 then come into direct contact with the urine absorbed in the absorbent material 2. If the absorbent material 2 comprises a scratch-proof layer of non-woven material, this has the additional advantage that inaccurate displays, which might occur as a consequence of possible contact with cat litter, are minimised or avoided. When the user pulls the indicator strip 1 out of the cat lavatory 8 completely, it is possible in this way to read off the urine levels found in a simple and hygienic manner.

The cat lavatory of the invention is particularly suitable for determining the sugar level, blood content and the like in the urine.

It goes without saying that a lavatory according to the invention can be used not only for cats, but also for dogs and other small animals.

The features of the invention disclosed in the above description, in the claims and in the drawings may be essential for carrying out the invention in its various embodiments both individually and in any combination.

1. A cat lavatory, comprising
at least one absorbent material comprising an insert of non-woven material, the insert having at least one indicator strip for determining a cat's urine levels, which is arranged on and/or in the absorbent material.

2. The cat lavatory as claimed in claim 1, wherein the cat lavatory comprises a container with a peripheral side wall, which is sealed on all sides in the condition in which it is delivered to the consumer, and which contains absorbent material, and which is designed such that it is opened before use and is disposed of in its entirety after use.

3. The cat lavatory as claimed in claim 2, wherein the container is a folded pack.

4. The cat lavatory as claimed in claim 1, further comprising a lower part and a replaceable pouch containing the absorbent material, which can be placed in the lower part.

5. A cat lavatory as claimed in claim 1, wherein the insert of non-woven material comprises a super-absorber and a scratch-proof layer of non-woven material disposed on top of the super-absorber.

6. The cat lavatory as claimed in claim 5, wherein a layer of litter is disposed above the scratch-proof layer of non-woven material.

7. The cat lavatory as claimed in claim 5, wherein the indicator strip is disposed between the super-absorber and the scratch-proof layer of non-woven material.

8. A cat lavatory as claimed in claim 1, wherein the indicator strip comprises at least one indicator field and a region for the user to handle the indicator strip.

9. The cat lavatory as claimed in claim 8, wherein the indicator field comprises at least one indicator point.

10. The cat lavatory as claimed in claim 8, wherein the indicator field, in the condition in which it is delivered to the consumer, is arranged within a protective cover.

11. The cat lavatory as claimed in claim 10, wherein the protective cover is made substantially from water-proof plastic material and is attached on and/or in the absorbent material.

12. The cat lavatory as claimed in claim 11, wherein the protective cover is attached on and/or in the absorbent material with spots of adhesive.

13. A cat lavatory as claimed in claim 1, wherein the indicator strip is substantially made from water-proof plastic material.

14. A cat lavatory as claimed in claim 1, wherein the indicator strip is fixed to the absorbent material with at least one further attachment means.

15. A cat lavatory as claimed in claim 1, wherein the indicator strip can be activated by pulling it out over the absorbent material.

16. A cat lavatory as claimed in claim 1, wherein the indicator field is disposed on the side of the indicator strip facing the super-absorber.

17. A method of determining a cat's urine levels using a cat lavatory as claimed in claim 1, comprising the step of: activating the indicator strip by drawning it over the insert of non-woven material.

18. The method as claimed in claim 17, wherein the indicator strip is used to determine urine levels, such as sugar and blood contents.

19. The method as claimed in claim 17, wherein the indicator is drawn over the super-absorber.
